(19) United States
(12) Patent Application Publication
(10) Pub. No.: US 2005/0165329 A1
(43) Pub. Date: Jul. 28, 2005

(54) MULTIPLE BIOPSY COLLECTION DEVICE

(75) Inventors: Thomas V. Taylor, Houston, TX (US); Frank G. Weeden, Houston, TX (US)

Correspondence Address: LUNDEEN & DICKINSON, LLP, PO BOX 131144, HOUSTON, TX, 77219-1144, US

(73) Assignee: REFLUX CORPORATION, Houston, TX (US)

(21) Appl. No.: 10/904,068

(22) Filed: Oct. 21, 2004

Related U.S. Application Data
(60) Provisional application No. 60/481,939, filed on Jan. 22, 2004.
(60) Provisional application No. 60/521,348, filed on Apr. 6, 2004.

(51) Int. Cl. A61B 10/00

(52) U.S. Cl. 600/566; 606/167; 600/567

(57) ABSTRACT

A biopsy device for taking multiple tissue samples from a single insertion point is described herein. The present device comprises a first elongated outer tube disposed about a second elongated inner tube, whereby the outer tube is adapted to slide axially in relation to the inner tube. The inner tube further includes a sample recess for obtaining samples, and the outer tube further includes a sample storage area for the storage of multiple samples during the sampling procedure. The sample storage area further includes means for securing each individual sample.

### CROSS REFERENCE TO RELATED APPLICATIONS

This application claims priority to U.S. Provisional Application Ser. No. 60/481,939 filed in the United States Patent and Trademark Office on Jan. 22, 2004, and U.S. Provisional Application Ser. No. 60/521,348 filed in the United States Patent and Trademark Office on Apr. 6, 2004.

### BACKGROUND OF THE INVENTION

This invention generally relates to biopsy instruments. More particularly, this invention relates to biopsy instruments especially useful for taking multiple tissue samples through a single insertion point, thereby minimizing the number of invasive medical procedures necessary to obtain the tissue samples for examination.

Often, it is either desirable or necessary to obtain specimens of tissue from humans and other animals, particularly in the diagnosis and treatment of patients with cancerous tumors, premalignant conditions, and other diseases or disorders. For example, in the case of cancer, when it is discovered that suspicious conditions exist, either by means of x-ray or ultrasound imaging, the physician typically performs a biopsy to determine if the suspicious cells are cancerous.

A biopsy can be done either by an open or percutaneous technique. Open biopsy is an invasive procedure using a scalpel, whereby either a portion (incisional biopsy) or the entire mass (excisional biopsy) is removed. Percutaneous biopsy is usually done with a needle-like instrument through a relatively small incision, and can be performed by fine needle aspiration (FNA) or through the taking of a core biopsy sample. In FNA biopsy, individual cells or clusters of cells are obtained for cytologic examination and can be prepared such as in a Papanicolaou smear. In a core biopsy, a core or fragment of the tissue is obtained for histologic examination.

Intact tissue from the organ, lesion, or tumor is preferred by medical personnel in order to arrive at a definitive diagnosis regarding the patient's condition. In most cases only part of the tissue in question needs to be sampled. The portions of tissue extracted must be indicative of the organ, lesion, or tumor as a whole. Often, multiple tissue samples from various locations of the mass being sampled may be taken.

The percutaneous biopsy procedure can be performed utilizing various techniques and devices. One such biopsy device can include an inner stylet positioned inside an outer cannula, whereby the stylet is able to slide into and out of the cannula. The stylet can be a solid, pointed needle having a tissue sampling recess, and the cannula can be a hollow, open-ended needle having a sharp tip. The stylet and cannula can be manipulated cooperatively to capture a tissue sample in the sample recess. Such existing devices can be manually operated, semi-automated, and automated.

Generally, manual biopsy devices allow for hand-controlled movement of either the stylet, the cannula, or both. Initially, the stylet and the cannula are inserted into soft tissue with the cannula covering the stylet tissue recess. The sytlet can then be manually advanced into the soft tissue to expose the tissue recess and the tissue prolapses into the stylet recess. The cannula can then be manually advanced to sever the tissue and capture a tissue sample, which can be held securely within the recess area of the stylet. The sample can then be withdrawn from the patient so that the tissue sample can be removed from the device, either by removing the stylet or the entire biopsy device.

Existing biopsy devices have been designed to be either reusable or disposable. The reusable devices can include a reusable handle portion with a disposable needle assembly. The stylet and cannula can be removed from the handle portion of the device and replaced with a new stylet and cannula. The handle can be cleaned, resterilied after use, and reused. Disposable devices generally include a permanently affixed stylet and cannula and are not re-sterilizable and therefore not reusable. Disposable devices can be used on one patient and then discarded.

Examples of traditional biopsy devices are disclosed in U.S. Pat. Nos. 4,600,014; 4,944,308; 4,958,625; and 5,368,045. Several existing devices have attempted to develop ways to take multiple tissue samples without having to withdraw and reinsert the biopsy device after each individual sample has been taken. In U.S. Pat. No. 6,554,779, to Viola et al. and U.S. Pat. No. 6,050,955 to Bryan et al., a device is demonstrated that is inserted directly into the patient to take multiple tissue samples through a single insertion, whereby the individual tissue samples are withdrawn from the device after they have been taken, while the device remains inserted in the patient.

Many prior art biopsy devices are delivered to the sampling site through an endoscope and lack a sharp tip or head designed to penetrate skin and/or tissue. Devices designed to take multiple samples, without having to be withdrawn through the endoscope after each sample is taken, are disclosed in U.S. Pat. Nos. 5,562,102 and 6,419,640 to Taylor. These devices provide a pincher type mechanism for obtaining each sample, with the successive samples pushing the prior sample rearward. The samples can be withdrawn from the devices by removing the stylet while the device remains inserted in the patient's body. Examples of similar devices include U.S. Pat. No. 6,142,957 to Diamond et al., U.S. Pat. No. 6,149,607 to Simpson et al., and U.S. Pat. No. 5,810,744 to Chu et al.

To minimize both the invasive nature of the procedure and patient discomfort, it is desirable to have a device capable of taking multiple samples, without having to remove and reinsert the biopsy device after each tissue sample is collected. The present invention satisfies these and other needs. Other aspects and advantages of the present invention will become apparent after reading this disclosure, including the claims, and reviewing the accompanying drawings.

### SUMMARY OF THE INVENTION

The multiple biopsy instrument according to the present invention allows the physician to take multiple separate and discrete tissue specimens in an ordered, identifiable fashion per single insertion of the device into the patient's body. By eliminating the requirement to withdraw the instrument after each sample has been taken, retrieve the sample from the device, and reinsert the device in either rthe same or an alternate location for another sample, the time spent for the procedure and the discomfort for the patient can be reduced. In addition, by making the present biopsy instrument disposable, rather than from non-disposable materials such as stainless steel, the need for sterilization of the instrument after each use is eliminated.

A preferred embodiment of the present invention provides a biopsy device for obtaining multiple tissue samples, comprising an inner elongated tubular body and an outer elongated tubular body, wherein the inner elongated tubular body includes first and second end portions and defines a hollow passage therein. The first end portion of the inner elongated tubular body includes a tip portion adapted to penetrate tissue and a sample recess adjacent to the tip portion, and the second end of the inner elongated tubular body comprises a handle portion. The outer elongated tubular body includes first and second end portions, and the inner elongated tubular body is adapted to move slidably within the outer elongated tubular body. The first end portion of the outer elongated tubular body can include a cutting edge and a sample storage space and the second end portion of the outer elongated tubular body can include a handle portion. The sample storage space includes means for securing the samples located within the storage area.

The invention can further include barbs located on the exterior of the inner tubular body for securing samples. The invention can further include barbs located on the interior of the outer tubular body for securing samples. The barbs can be angled toward the handle of the device.

The invention can further include a plurality of holes located within the sample recess, wherein the holes communicate with a vacuum source. The invention can further include a sharpened tip portion to the device, whereby the tip portion is adapted to penetrate skin and tissue of a human or animal patient. The tip portion can be connected to the device through screw mounted threads or some other detatchable means.

The device can be constructed of a substantially rigid material, and can be metallic, ceramic, plastic, or a combination thereof. The device can be disposable, or the device can be non-disposable thus necessitating sterilization between each individual patient.

The device can include graduated markings on the second end portion of the inner elongated tube, and the device can also include graduated markings on the first end portion of the outer elongated tube.

The present invention also provides a method for obtaining multiple tissue biopsies of an organ comprising the sequential steps of: (a) inserting the multiple sample biopsy device into a mass or organ to be sampled; (b) positioning the sampling end of the device at the location to be sampled; (c) retracting the outer tube to expose a sample recess of the inner tube and allowing the tissue to prolapse into the sample recess; (d) distally extending the outer tube bringing the cutting end of the outer tube in contact with the sample mass and securing the sample within the device; (e) engaging, e.g. by transfixing, the sample mass on the barbs located in the sample storage area; (f) repeating steps (c) through (e) to sequentially collect a plurality of tissue samples in the sample storage area; and (g) withdrawing the biopsy device from the patient's body. The tissue samples can be removed from the device for examination. The method can also include reading the graduation markings on the inner and outer tubes for identifying the tissue location and for controlling the amount of the sample recess exposed to the mass or organ being sampled, or for controlling the depth at which the device is inserted within the patient.

### BRIEF DESCRIPTION OF THE DRAWINGS

FIG. 1 is a side cross-sectional view of a multiple biopsy device of one embodiment of the present invention.

FIG. 2 is a side cross-sectional view of the device engaging a sample.

FIG. 3 shows multiple side cross-sectional views of the as the sample is taken and stored in the sample storage area.

FIG. 4 is a side cross-sectional view of the handle end of a multiple biopsy device of one embodiment of the present invention.

FIG. 5 is a radial cross-sectional view of the embodiment of the device shown in FIG. 2, along the lines 5-5.

### DETAILED DESCRIPTION OF THE INVENTION

The multiple biopsy device of the present invention is suitable for taking multiple biopsy samples through a single insertion point. Use of the present device reduces equipment usage and operating time for the biopsy procedure, while at the same time lessening patient discomfort through the elimination of repeated insertion, extraction, and reinsertion procedures by streamlining the procedure to a single insertion during which multiple biopsy samples of an affected area are taken. Although the present invention can be made in many different forms, the preferred embodiments are described in this disclosure and shown in the attached drawings. This disclosure exemplifies the principles of the present invention and does not limit the broad aspects of the invention only to the illustrated embodiments.

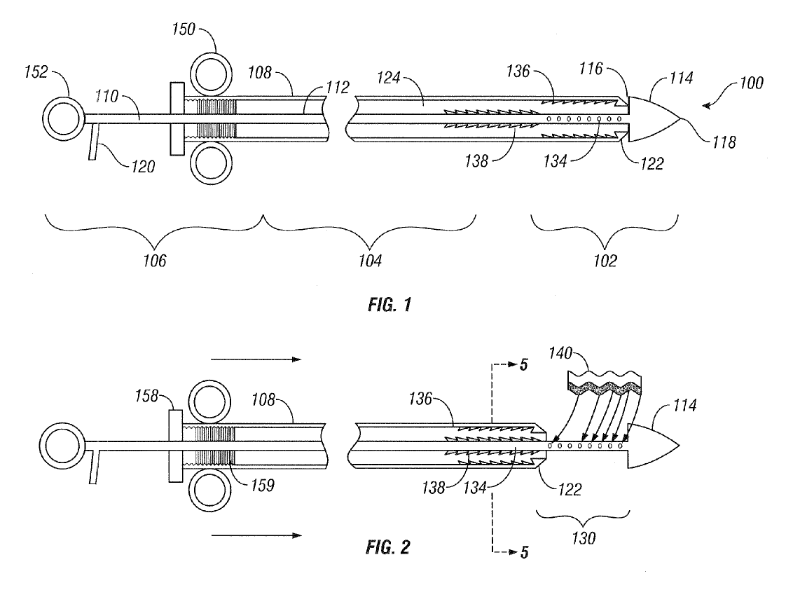

Referring now in specific detail to the drawings, in which like reference numbers identify similar or identical elements throughout the several views, and initially to FIGS. 1 and 2, a biopsy instrument constructed in accordance with certain aspects of the present disclosure is shown generally as apparatus 100. The biopsy device can be used to obtain multiple samples of tissue per insertion of the biopsy device into a diseased or affected area of the patient's body desired to be sampled. In general, the instrument can include a tip or first end portion 102, an elongated body portion 104, and a handle portion 106. FIG. 1 shows a perspective view of one embodiment of the biopsy instrument 100 made in accordance with the principles of the present invention. The biopsy instrument 100 can include an outer cannula 108, slidably positioned about inner stylet 112. Stylet 112 consists of a tip 114, a tube 110 extending the length of biopsy device 100, and a handle portion 152.

The stylet 112 includes a sharpened tip 114 designed to penetrate the body of a human or animal. Tip 114 consists of a base 116 which extends outward to a diameter equal to or greater than the diameter of cannula 108, and then tapers to a sharp point 118. Tip 114 can be preferably constructed of a metallic compound or alloy, and can be detachably affixed to stylet 112, but can also be constructed of a hardened ceramic or polymer material. As will be apparent to those of skill in the art in view of the disclosure, the tip portion of the instrument can be readily adapted to attach to either a relatively rigid or a relatively flexible inner tube, of varying lengths and diameters, depending on the desired application of the device. Similarly, the tip can be of varying lengths and diameters, depending upon the desired application. The method of attachment of tip 114 to stylet 112 can preferably be screw mounted, or can be any other mounting system that allows for removal of the tip from the body of the stylet. The tip 114 of stylet 112 is preferably adapted to enable insertion of the biopsy device with a minimum amount of discomfort to the patient.

The end of stylet 112 has a sample recess 130 located adjacent to tip 114. Sample recess 130 can include a plurality of holes 134 located at the distal end, which can communicate with an attached vacuum device (not shown) through hollow tube 110. The hollow tube connects sample recess holes 134 to the vacuum source through inlet 120. Inlet 120 can preferably be located at the handle portion 106 of the device 100. The sample recess can be arcuate, with a plurality of holes located on one side of the stylet, or it can be cylindrical with a plurality of holes disposed circumferentially about the sample recess.

Cannula 108 and stylet 112 are slidably positioned relative to each other whereby the cannula of biopsy device 100 can be manipulated, thereby opening and closing a sample recess 130 located at the distal end of the stylet. In similar fashion, manipulation of the cannula 108 and stylet 112 performs the task of severing the tissue sample. The cannula 108 is a hollow elongated tube having a sharpened and open-end portion 122 and a handle portion 150. The sharpened end of the cannula 122 preferably extends about the circumference of the distal end of the cannula and preferably is adapted to sever the tissue sample cleanly and efficiently from the mass being sampled as the sharpened end 122 contacts the base 116 of the tip 114.

The vacuum inlet 120 can be located on the handle portion 106 of stylet 112, and the vacuum can be provided by an attached vacuum source, including but not limited to, vacuum pumps or hand-operated syringes. Vacuum inlet 120 can optionally have an attached stopcock or release valve (not shown) for fine control over the application of suction to the stylet and sample recess.

Vacuum suction can be applied to the sample recess 130 through the hollow passage 110 within stylet 112, and can be communicated to the tissue sample through a plurality of holes 134 located within the sample recess 130. The vacuum source can preferably be applied after the tip of the device has been positioned and the sample recess exposed to the mass being sampled. As shown in FIG. 2, the vacuum communicated through a plurality of holes 134 draws the tissue sample 140 toward the sample recess 130. In FIG. 2, the sample is shown as being drawn to one side of the sample recess, but tissue also can be drawn circumferentially into the recess. Suction holds the tissue sample in place within sample recess 130, until the blade portion 122 of cannula 108 severs the tissue sample from the target mass.

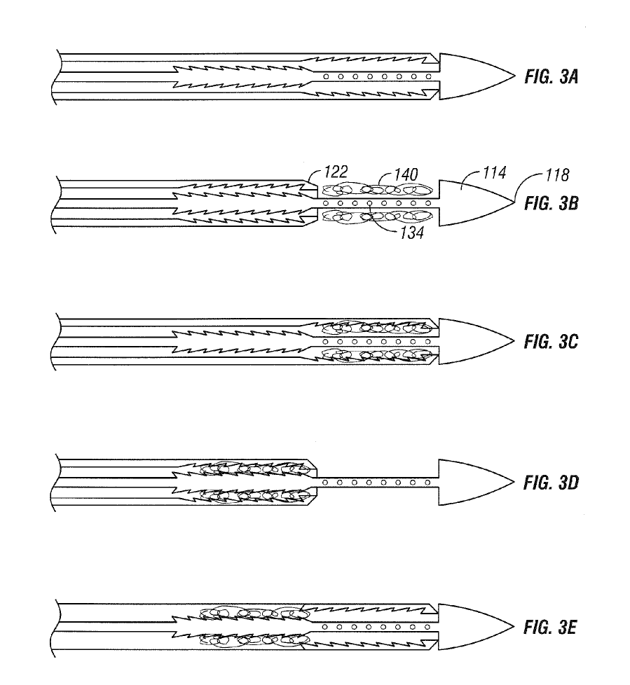

FIG. 3(a) through (e) demonstrates the use of the biopsy device to obtain multiple samples through a single insertion point. FIG. 3(a) shows the relative positions of the stylet and cannula during insertion and positioning. FIG. 3(b) shows the retraction of the cannula, to expose the sample recess. FIG. 3(c) shows the closing of the sample recess after cutting of the tissue sample and the barbs on the interior of the cannula as they engage the tissue sample. FIG. 3(d) shows the cannula prepared for the next sample as the sample recess is exposed to the tissue being sampled, and the first tissue sample has been repositioned on the barbs located in the storage space of the stylet 112. FIG. 3(e) demonstrates how the stored samples remain in place in the storage space.

To cut the tissue sample, the cannula 108 is extended distally toward the tip of stylet 112, bringing the blade portion 122 of the cannula in contact with the sample mass 140. The blade portion 122 is extended until it contacts the tip base 116, whereby the tissue sample is completely severed from the mass being sampled. Optionally, cannula 108 can rotate about the axis of the biopsy device, thereby providing an additional means for cutting the sample. As the tissue sample 140 is cut by the blade portion 122 of the cannula 108, the sample can be contacted by a series of barbs 136 located on the interior wall of the cannula 108, extending rearward from the blade end 122. Preferably, the barbs extend rearward from the blade portion 122 for a distance equal to the length of the sample recess 130. A second set of barbs 138 can be located on stylet 110, extending rearward proximal to the sample recess 102, preferably beginning at a point overlapping the barbs 136 located on the interior of the cannula 108. Similar to the first set of barbs 136, the second set of barbs 138 located on the stylet 112 extend rearward from the tip portion 102 of the biopsy device 100 toward the handle portion 106 of the device, preferably being located rearward from the sample recess 130.

The samples can each be stored in a sample storage area 124, located within cannula 108 and exterior to stylet 112. The barbs can be angled rearward toward the handle portion 106 of the biopsy device to hold the individual samples in place when the cannula 108 is retracted and the sample recess 130 is exposed for the next sample. The barbs can preferably be positioned in a manner such that the manipulation of the device 100 does not rip and/or tear the stored biopsy samples. The samples stored within storage area 124 of the device 100 are thus prevented from being released, thereby blocking the sample recess 130.

The barbs in the sample storage area 124 can be engaged through the normal operation of the device in obtaining tissue samples. During operation of the device, to obtain the first tissue sample, the cannula 108 is retracted, thereby exposing the sample recess area 130. The tissue sample can be received within sample recess 130, preferably by means of applied suction communicated through the plurality of holes 134 in the sample recess. While the suction is being applied, the cannula 108 is extended, engaging the blade portion 122 of the cannula 108, severing the tissue sample. As the cannula blade portion 122 cuts the tissue sample, the sample is brought to the sample storage area where the barbs 136 located within the interior portion of the cannula 108 engage the tissue sample. When the cannula 108 is retracted for the next sample, the stylet barbs 138 located on the exterior of stylet 112 then engage the first tissue sample, as the barbs 136 on the cannula 108 slide away from the stored tissue sample. As each successive sample is obtained, the samples within the storage area 124 are each pushed toward the handle end 106 of the device 100 by the new sample and the manipulation of the biopsy device. Because the barbs 136,138 that hold the sample are angled toward the handle portion 106 of the biopsy device 100, the samples within the sample storage 124 can slide easily toward the rear of the device during the manipulation of cannula 108. The barbs 136, 138 prevent movement of the stored samples forward, toward the tip portion 102 of the device 100, thereby preventing the stored sample from interfering with the collection of successive samples by blocking the sample recess 130. Thus, once within the sample storage area 124, samples can only move rearward toward the handle portion 106 of the biopsy device 100.

The barbs 136, 138 can be disposed lengthwise from the tip portion 102 to the handle portion 106 of the device in the exterior of the stylet 112 and the interior of the cannula 108. Alternatively, the barbs 136, 138 can be arranged as shown in FIGS. 1 and 2 wherein the first set of barbs 136 is equal in length to the sample recess 130 on the stylet 112 and extend rearward from holes 134 for a length approximately equal to the sample recess 130. Where the barbs extend rearward for a fixed length, the contact of the sample with the barbs is limited.

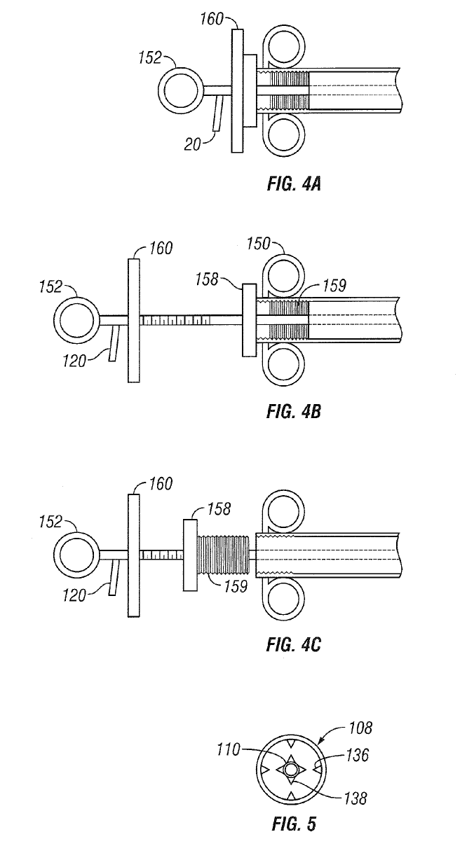

Generally, handle portion 106 consists of two separate hand operated cooperative finger rings. Cannula handle 150 attaches to the proximal end of cannula 108, opposite the sampling, or tip, end of the device. Cannula handle 150 can consist of at least one, and preferably two, finger rings or other external means for controlling the movement of the outer cannula, located at the proximal end of the cannula 108 allowing the operator of the device 100 to extend and retract the cannula with one hand while the finger rings can be helpful in the manipulation of the biopsy device, they are not required. The stylet handle 152 can consist of a single ring located at the proximal end of the stylet, opposite to the tip end 102 of the device 100. Both the cannula handle 150 and the stylet handle 152 are positioned on the device to allow the operator to apply force during the insertion of the device 100 and the cutting of the samples along the axis of the device. Movement of the handle portion 106 of the device 100 during the sampling process is demonstrated in FIGS. 4(a), (b), and (c). In FIG. 4(a), the handle portion of the cannula is shown fully retracted, thereby exposing the sample recess corresponding to FIG. 3B. In FIG. 4(b), the handle portion of the cannula 108 is shown fully extended, corresponding to FIG. 3C wherein the blade portion of the cannula can be in contact with the sample mass, severing tissue sample, and closing the sample recess. In FIG. 4(c) the retaining screw is removed from the cannula, allowing the stylet and retained samples to be removed from the device.

Cannula handle 150 is designed be operated with a single hand, thus allowing for the physician the free use of his alternate hand to control the vacuum application or to perform any other necessary task during the procedure. The particular handle 150 illustrated herein is considered exemplary, although any of a wide variety of handle designs allowing for control over the device can be employed.

The proximal end of stylet 112 can optionally include graduated markings, using dimensions such as millimeters or centimeters, along the axis of the device extending toward the handle portion 106 of the device, allowing the physician to determine the depth at which either the tip portion 102 has been inserted or the amount of sample recess 130 exposed as the tip is inserted in the mass being sampled. The stylet handle 152 can also include a hilt 160, located at a point that allows a maximum length of sample recess 130 to be exposed without having to read the graduated markings on the device. The stylet 112 can be secured and centered within the device by means of a retaining cylinder 158 attached at the handle end of the cannula 108. The retaining cylinder 158 can include external threads 159 and can attach to cannula 108 via a screw mounting to interior threads of cannula 108, as shown in FIG. 4(c). The retaining cylinder 158 ensures that stylet 112 remains centrally positioned within the cannula 108. The retaining cylinder 158 can include a central passage providing slidable manipulation of the stylet 112 in operation of the device 100. Similaroly, the tip end of the cannula can include graduated markings (not shown) for determining the depth the device is inserted within the patient's body.

FIG. 5 shows a cross-sectional view of one embodiment of the device and demonstrates the relationship of the inner stylet 112 to the cannula 108, and the barbs 136 and 138 located therein. The barbs 136, 138, as shown in FIG. 5, can be spaced at 90 degree intervals, however it is understood that the barbs may be spaced at different intervals, for example at 45 or 60 degree intervals, or may be located circumferentially throughout the interior of the device.

The barbs located on the cannula 108 and stylet 112 are preferably formed of a metallic compound or alloy, but can also be a medical grade plastic, polymer or composite material as well. The barbs can be sufficient in length to secure the tissue sample, while at the same time preferably preventing damage to the sample.

Once the desired number of biopsy samples have been taken, or if the storage space has been filled to capacity, the biopsy device can be removed from the body. To retrieve the individual samples, the tip of the device 114 is removed, either by unscrewing or detaching, and the retaining cylinder 158 is removed from the cannula 108, either by unscrewing or detaching by other means, thereby allowing the stylet 112 to be removed from the handle portion 106 and cannula 108 of the device. The tissue samples are removed from the device during removal of the stylet 112, and catalogued to the area of the organ or mass from which they were removed. Between patients, used disposable biopsy devices are discarded, and the used non-disposable biopsy device can be sterilized for reuse.

The biopsy device of the present invention can be constructed of a rigid metal material, such as stainless steel or an alloy material, enabling it to be re-sterilized and reused. Alternatively, the device can be constructed from a composite, ceramic, polymer, or plastic material, such as polypropylene, polyethylene, polyacrylic, or the like, and can be disposed of after use. Other embodiments of the invention can also be envisioned wherein a plurality of materials can be combined to form different components of a single instrument.

The present invention can be constructed in a variety of different sizes and dimensions, depending on the need of the physician. For use in obtaining breast biopsy samples, the device is preferably between 10 and 12 inches in length, from the tip portion 102 to the stylet handle 152. The base portion 116 of tip 114 and outer cannula 108 can preferably have a diameter of less than 6 mm (0.25 in.), more preferably 5 mm (0.2 in.) or less in diameter.

The present invention can also include means for providing an electrical current to the tip portion 102 of the device, thus enabling the physician or clinician to cauterize the area being sampled.

To use the biopsy instrument of the present invention taking multiple specimens for biopsy in a single insertion into the patient, the physician first locates the tissue mass to be sampled, and inserts the device into the patient, reading the graduated markings on the outer cannula to determine the depth at which the instrument has been inserted.

Once the tip of the instrument has been positioned in the desired location within the patient's body, the handle 150 of the outer cannula tube 108 is manipulated, sliding the cannula 108 longitudinally away from the tip portion 102 of the instrument, thus exposing the sample recess 130. If the vacuum source (not shown) has not already been applied, it can be applied at this time. Under applied vacuum, the tissue prolapses into the sample recess 130. The outer cannula 108 can then be manipulated by sliding the tube longitudinally forward engaging the blade portion 122 of the cannula 108 to the tissue mass, separating the tissue sample from the remaining mass. The outer cannula 108 can be manipulated until the blade portion 122 contacts the base 116 of stylet tip 114, thereby completing the severing of the tissue sample from the remaining sample mass. The biopsy instrument 100 can be repositioned within the patient without first removing the instrument and the sampling process can be repeated. Additional samples can be taken by repeating the steps detailed above.

Once the desired number of tissue samples have been taken, the biopsy instrument 100 can be removed from the patient's body. The tip of the stylet 114 can be removed from the stylet body 112 and the retaining cylinder 158 removed from the end portion of cannula 108. The stylet body 112 can be extracted from the instrument by retracting through the handle portion of the outer cannula 108. The individual tissue samples can be removed from sample storage area and cataloged for later examination.

The foregoing description of the present invention is illustrative and explanatory thereof. Various changes in the materials, apparatus, particular parts, and methods of use employed will occur to those skilled in the art. It is intended that all such variations within the scope and spirit of the appended claims be embraced thereby.

1. A biopsy device for obtaining multiple tissue samples, comprising:
an outer elongated rigid tubular body disposed about an inner elongated tubular body wherein the inner tubular body is adapted to move slidably within the outer tubular body;
the inner elongated tubular body comprising a first and second end portion and defining a hollow passage therein, the first end of the inner elongated tubular body adapted to take samples comprising a tip portion adapted to penetrate tissue and a sample recess proximal to the tip portion and the second end of the inner elongated tubular body comprising a handle portion;
wherein the outer elongated tubular body has a first and second end portion, the first end portion of the outer elongated tubular body adapted to cut the sample comprising a cutting edge and sample storage space proximal to the cutting edge, and the second end portion of the outer elongated tubular body comprising a handle portion;
wherein the sample storage space further comprises means for securing samples within the storage space.

2. The biopsy device of claim 1, wherein the securing means are barbs.

3. The biopsy device of claim 1, wherein the barbs are located on an outer surface of the inner tubular body.

4. The biopsy device of claim 1, wherein the barbs are located on an inner surface of the outer tubular body.

5. The biopsy device of claim 2, wherein the barbs are angled toward the handle portion of the device.

6. The biopsy device of claim 1 wherein the sample recess further comprises a plurality of holes adapted to communicate with a vacuum generating source.

7. The biopsy device of claim 1 wherein the tip portion includes a sharpened conical section adapted to penetrate the skin.

8. The biopsy device of claim 1 wherein the tip portion is connected to the inner elongated tubular body by screw threads.

9. The biopsy device of claim 1, wherein the device is constructed of a metallic material.

10. The biopsy device of claim 1 wherein the device is constructed of a substantially rigid plastic material.

11. The biopsy device of claim 1 wherein the device is made of a disposable material.

12. The biopsy device of claim 1 wherein the second end portion of the inner elongated tube includes graduation markings.

13. The biopsy device of claim 1 wherein the first end portion of the second elongated tube includes graduation markings.

14. A method for obtaining multiple tissue biopsies of an organ, comprising the sequential steps of:
inserting the biopsy device of claim 1 into a mass to be sampled;
positioning the sampling end of the device at a location to be sampled;
retracting the outer tubular body to expose the sample recess of the inner tubular body and allow the sample to prolapse into the recess;
distally extending the outer tubular body bringing the cutting end of the outer tube in contact with the sample mass and securing the sample within the device;
transfixing the sample mass on the securing means located in the sample storage area;
repeating steps (c) through (e) to sequentially collect a plurality of tissue samples in the sample storage area;
withdrawing the biopsy device from the body and removing the tissue samples for examination.

15. The method of claim 14, including reading graduation markings on the inner and outer tubes for identifying the tissue location and controlling the size of the sample.
